(19) United States
(12) Patent Application Publication
(10) Pub. No.: US 2025/0120651 A1
(43) Pub. Date: Apr. 17, 2025

(54) COMPUTER DEVICE FOR WEARING ON THE HUMAN BODY, AND SENSOR DEVICE

(71) Applicant: ONVY HealthTech Group GmbH, Grünwald (DE)

(72) Inventor: Julius RACHOR, München (DE)

(21) Appl. No.: 18/730,584

(22) PCT Filed: Jan. 11, 2023
(86) PCT No.: PCT/DE2023/100016

§ 371 Date: Jul. 19, 2024

(30) Foreign Application Priority Data
Jan. 21, 2022 (DE) 10 2022 101 445.2

(51) Int. Cl. A61B 5/00; A61B 5/0205; A61B 5/024; A61B 5/0531

(52) U.S. Cl. CPC A61B 5/6826; A61B 5/0205; A61B 5/02416; A61B 5/02438; A61B 5/0531; A61B 2562/0214

(57) ABSTRACT

Proposed is a ring-shaped computer device for wearing on the human body, in particular on a finger, which computer device is configured to continuously collect vital parameters and/or bioactive markers of a wearer and make them available to a connected device and has a ring-shaped main body with an at least partially optically transparent inner surface which faces the wearer's skin, a first sensor for optically detecting vital parameters, in particular a heart rate, via the wearer's skin, wherein the first sensor is separated from the wearer's skin by at least one optically transparent detection area of the inner surface, and a second sensor for determining electrical properties of the wearer's skin by means of electrical contacts, wherein the electrical contacts are applied to the optically transparent inner surface of the main body.

### FIELD OF THE INVENTION

The invention relates to a ring-shaped computer device for wearing on the human body, in particular on a finger, and to a sensor device for recording vital parameters and/or bioactive markers on the human body.

### BACKGROUND

Wearable computer devices are known from the state of the art (e.g. from US 2016/0213267 A1 or US 2015/0220109 A1), which continuously vital collect parameters and/or biomarkers of a wearer on a finger and make them available to the user via connected devices. The wearable computer devices use various sensors to record the wearer's mental and physiological state. In particular, the devices utilise optical sensors for heartbeat measurement, acceleration sensors and gyroscopes for time-dependent positioning of the computer device in space, at least one temperature sensor and also at least one sensor for measuring the emotional and physiological state of the wearer via the electrical properties of the wearer's skin.

If an optical sensor is used for heartbeat measurement, this requires an interference-free optical measurement path between the sensor and the wearer's skin, at least in the wavelength range of the light emitted by the sensor. In the current state of the art, this measurement path is realised via an opening in the surface of the wearable computer device. This opening is transparent at least for the wavelength range of the light emitted by the optical sensor. This solution increases the complexity of the manufacture of the device and may also mean that the opening cannot be sealed airtight and watertight. Further embodiments of the prior art are such that at least the part of the housing of the wearable computer device facing the body of the wearer is formed as an optically transparent component. In order to integrate a sensor for detecting electrodermal activities in addition to an optical sensor, the state of the art also proposes further openings in the housing surface of the device in order to introduce electrically conductive contacts into the surface. This again leads to greater component complexity as well as potential air and water permeability.

The problem addressed by the invention is that of at least mitigating these disadvantages.

### BRIEF DESCRIPTION OF THE INVENTION

This problem is solved by the invention specified in the independent claims. Advantageous further embodiments can be found in the dependent claims.

The invention creates a ring-shaped computer device for wearing on the human body, in particular on a finger, which is configured to continuously collect vital parameters and/or bioactive markers of a wearer and make them available to a connected device, which computer device a ring-shaped main body with an at least partially optically transparent inner surface which faces the wearer's skin, a first sensor for optically detecting vital parameters, in particular a heart rate, via the wearer's skin, wherein the first sensor is separated from the wearer's skin by at least one optically transparent detection area of the inner surface; and a second sensor for determining electrical properties of the wearer's skin by means of electrical contacts, wherein the electrical contacts are applied to the at least partially optically transparent inner surface of the main body.

According to the invention, the non-conductive transparent inner surface of the computer device can be used to carry electrical sensor contacts of an EDA sensor. The inner surface of the computer device may have at least one optically transparent area which is used to detect the heart rate by the optical sensor. The electrical contacts can be applied on the inner side of the ring-shaped computer device directly to the at least partially transparent area, which can also be used at least partially by an optical sensor. The electrical contacts can be arranged or formed in such a way that they do not obstruct the measurement path of the optical sensor. The inner surface of the ring-shaped sensor offers sufficient space to arrange electrical contacts on it that can safely contact the wearer's skin. In this embodiment, the optically transparent main body is a component that forms at least part of the outer housing of the wearable computer device. This main body is formed in such a way that it is largely transparent at least for electromagnetic radiation of the wavelengths required for the measurement principle of the heartbeat sensor (PPG). In order to enable measurement of the electrodermal activity (EDA) or the (psycho) galvanic skin reaction of the wearer, two galvanically separated electrical contacts are applied to the surface of this main body. This makes it possible to embody the surface or part of the surface of the device as a transparent component, while at the same time enabling the measurement of the wearer's electrodermal activity. No relatively large openings in the surface are required and the computer device can be hermetically sealed.

In one embodiment of the invention, the electrical contacts are formed to be in contact with the wearer's skin, and the computer device further comprises internal components such as a printed circuit board for receiving the sensors and a data interface, a processor and at least two battery modules for supplying power to the printed circuit board and the processor, which are interconnected to form an overall battery.

The electrical contacts can be part of the EDA sensor, which comprises further components that are arranged together with further components inside the computer device. For example, and not exhaustively, the computer device may comprise a printed circuit board on which the optical sensor and/or at least part of the EDA sensor are arranged. Furthermore, a processor and a data interface may be connected to or arranged on the printed circuit board. The printed circuit board can be embodied to be flexible in order to adapt to the ring-shaped form of the computer device. The processor can receive the raw data from the sensors, process it and forward it via the data interface (e.g. Bluetooth®) to a connected device, such as a smartphone and/or a server. The computer device may further comprise one or more battery modules to power the electrical components. Several battery modules can be interconnected to form an overall battery.

In one embodiment of the invention, the electrical contacts are formed by an electrically conductive thin film, and/or the main body is optically transparent and the electrical contacts are applied to the optically transparent main body. In this embodiment, the optically transparent main body is a component which forms at least part of the outer housing of the wearable computer device. The electrical contacts are applied to a surface of the main body using thin-film technology. The electrically conductive layers forming the contacts can be applied, for example, by chemical vapour deposition, physical vapour deposition or galvanic processes, as well as by a combination of these or other processes. This makes it possible to form the surface or part of the surface of the device as a transparent component, while at the same time enabling the measurement of electrodermal activities of the wearer.

In particular, the main body is formed so that it is transparent to electromagnetic radiation of the wavelengths required for the measurement principle of the heart rate sensor, in particular a photoplethysmogram (PPG).

In one embodiment of the invention, the electrical contacts are arranged distributed over a large area over the entire inner surface of the main body, and the detection area of the first sensor is not covered by the electrical contacts. In particular, the electrical contacts can be formed with a very large surface area, i.e. with the exception of the detection area (i.e. the minimum required free optical measurement path of the optical sensor) they can occupy the entire inner surface of the computer device. This leads to improved contact between the wearer's skin and the EDA contacts. This is especially true when the wearer of the ring is moving and only parts of the inner surface of the housing, i.e. the inner surface of the device, are temporarily in contact with the wearer's skin.

In one embodiment of the invention, the electrical contacts are formed in a meandering shape on the transparent inner surface of the main body, and/or the computer device comprises two groups of electrical contacts which are arranged distributed over the inner surface.

The contacts can be formed in a meandering or serpentine shape on the inner surface. This ensures that the wearer's skin is reliably contacted, i.e. there are sufficiently large contact possibilities, but material can be saved compared to a contact formed as a surface. Furthermore, the electrical contacts can each comprise several groups of sub-contacts, wherein the sub-contacts of each group are electrically connected to each other, and wherein the groups are galvanically separated from each other. The sub-contacts of each group can be arranged here intermittently with the sub-contacts of the other group. It is also possible to form “contact centres” by arranging the sub-contacts of a group in a cluster. This can help to improve contact with the skin in specific sports where, for example, one-sided loads act on the computer device and skin contact on the opposite side is therefore limited.

In one embodiment of the invention, the computer device comprises a via in the inner surface of the main body for contacting the internal electronics, in particular the printed circuit board. The via can be only a very small hole through the inner side of the main body. However, if this leads the contacts into the interior of the main body, it is completely closed, e.g. by electroplating. This means that larger openings can be avoided and the interior of the main body remains hermetically sealed.

In one embodiment of the invention, the computer device comprises a guide for the contact connections via an end face of the main body into the interior of the main body for contacting the internal electronics, in particular the printed circuit board, by means of the electrical contacts. The contact connections can also initially be guided away from the inner surface to an end face of the main body. This avoids holes on the inner side and further improves the tightness.

According to a further aspect of the invention, a sensor device is provided for recording vital parameters and/or bioactive markers on the human body, comprising a first sensor for optically detecting vital parameters, in particular a heart rate, comprising at least one optically transparent detection and a sensor for area; second determining electrical properties of the wearer's skin by means of electrical contacts, wherein the electrical contacts are at least partially applied to the optically transparent detection area of the first sensor.

All embodiments of the computer device according to the invention are also transferable to the second aspect of the invention.

### BRIEF DESCRIPTION OF THE DRAWINGS

Exemplary embodiments of the invention are described in greater detail below with reference to the accompanying drawings, in which

FIG. 1 shows an exemplary embodiment of a computer device with internal electrical contacts according to the invention;

FIGS. 2a and 2b are drawings showing details of various exemplary sensor arrangements on the inner surface of the computer device according to the invention;

FIG. 3 shows a further embodiment according to the invention;

FIG. 4 is a drawing showing a detail of a further embodiment according to the invention;

FIGS. 5a to 5c show exemplary sensor arrangements on the inner surface of the computer device according to the invention.

### FIGURE DESCRIPTION

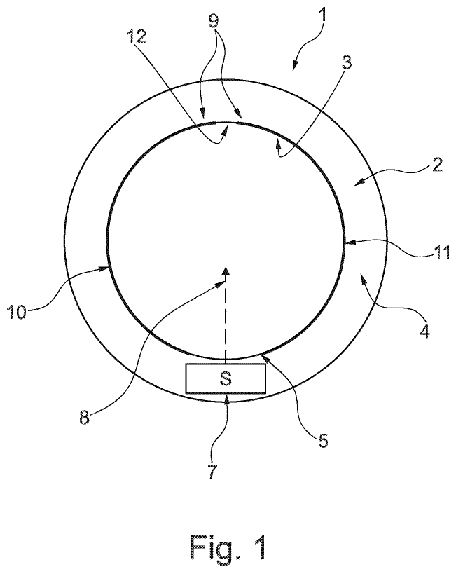

FIG. 1 shows a computer device 1 in the form of a finger ring according to the invention. The computer device 1 comprises a main body 2 with an inner surface 3 and an end face 4. The inner surface 3 is formed in one piece and is at least partially optically transparent. The inner surface 3 comprises a detection area 5, which is required at least in the range of wavelengths for the optical detection of a heart rate of the wearer by a photoplethysmogram, PPG. A sensor assembly 7, which comprises a PPG sensor and at least parts of an EDA sensor, is arranged in an interior space 6 of the computer device 1. The sensor assembly 7 is arranged below the detection area 5 to enable the PPG sensor to detect the heart rate via a measurement path 8. Electrical contacts 9 are applied to the inner surface 3 and are galvanically spaced apart. In the example shown, the electrical contacts 9 are formed as thin, electrically conductive layers. A first electrical contact 10 is applied to a first half of the inner circumferential surface (inner surface 3) and a second electrical contact 11 is applied to a second half of the inner surface 3. The contacts 10, 11 are spatially and galvanically spaced apart and electrically isolated from each other in the region of the detection area 5 and also in an area 12 opposite the detection area 5. The electrical contacts 10, 11 are formed by a thin layer and are arranged distributed over a large area over the respective halves of the inner surface 3. The detection area 5 is not covered by the contacts 10, 11. If contacts 10, 11 are used that are not provided over the entire area, but rather are in track form, in particular meandering, the detection area can also be provided with contacts, or can be reduced to a minimum, which is necessary to obtain the measurement path 8.

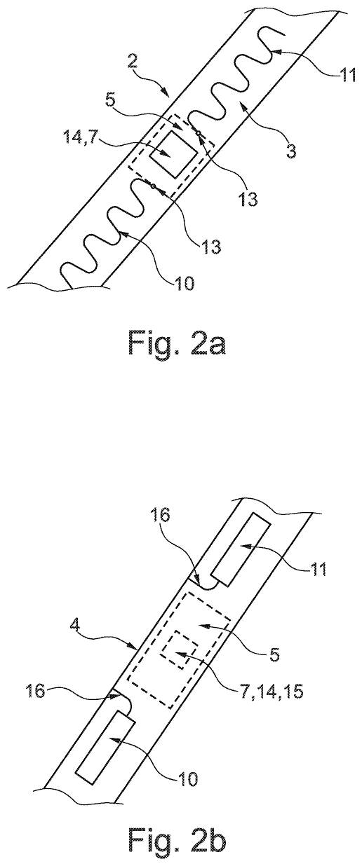

FIG. 2a shows a possible arrangement of the electrical contacts 9 on the inner surface 3 of the computer device 1. The electrical contacts 10 and 11 are applied to the inner surface 3 of the main body 2 as meandering tracks and are galvanically separated. The contacts 10, 11 are guided into the interior 6 of the main body 2 by means of vias 13. The vias 13 are located in an area surrounding the sensor assembly 7 and can also be located within the optically transparent detection area 5 of the optical sensor 14. The vias 13 are connected to at least one second sensor 15 (EDA sensor) (not shown).

FIG. 2b shows a further possible arrangement of the electrical contacts 9 on the inner surface 3 of the computer device 1. The contacts 10, 11 are guided to the end face 4 of the main body 2 via a guide 16.

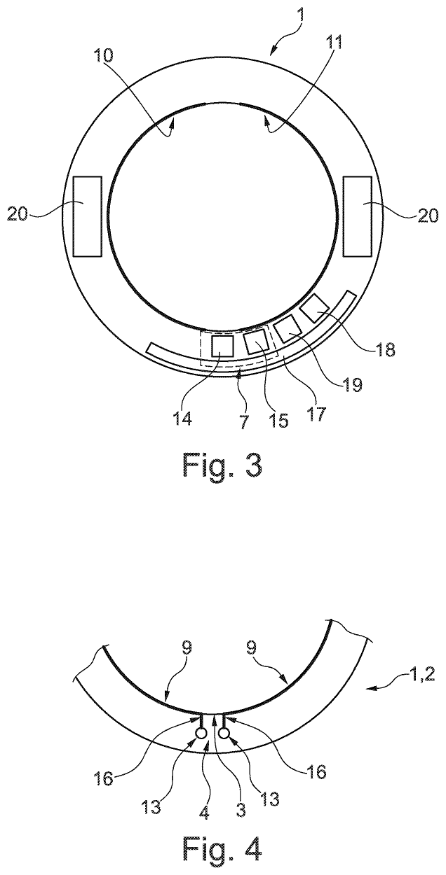

FIG. 3 shows a further embodiment according to the invention. In addition to the electrical contacts 10, 11 and the sensor assembly 7 (outlined by a dashed line), the computer device 1 comprises a printed circuit board 17. The printed circuit board 17 is connected to the sensor assembly 7. A processor 18 and a data interface 19 are arranged on the printed circuit board 17. The sensor assembly 7 comprises the optical sensor 14 and the EDA sensor 15. Furthermore, at least two battery modules 20 are arranged in the interior 6 of the main body 2 and are interconnected to form an overall battery.

FIG. 4 shows a further possible arrangement of the electrical contacts 9 on the inner surface 3 of the computer device 1. The contacts 10, 11, which are formed as a flat, thin layer, are guided via the guide 16 to the end face 4 of the main body 2, where they are guided into the interior 6 of the main body 2 by means of vias 13 and connected to the sensor assembly 7, in particular to the EDA sensor 15.

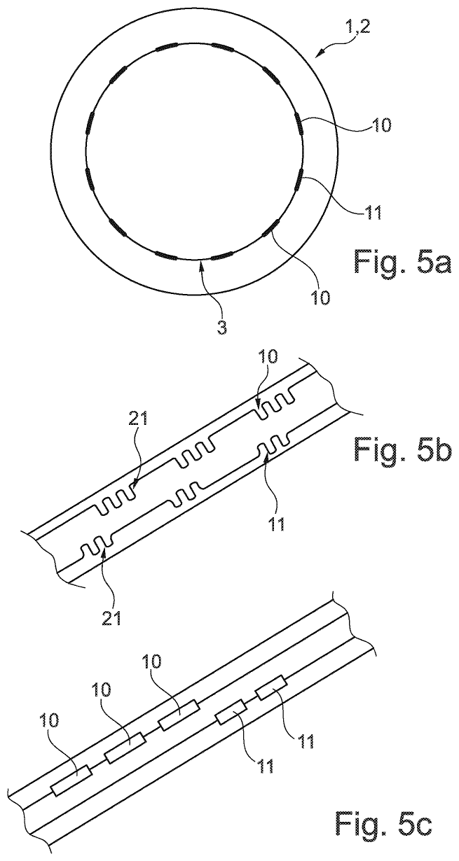

FIGS. 5a to 5c show further exemplary arrangements of the electrical contacts 9 on the inner surface 3 of the computer device 1. The first and second contacts 10, 11 are arranged alternately along the inner circumferential surface.

FIG. 5b shows an embodiment in which the contacts 9 are formed in a meandering fashion and run side by side as tracks on the inner surface 3. The first and second contacts 10, 11 each have serpentine portions 21, which are arranged offset.

FIG. 5b shows an embodiment in which the contacts 9 comprise groups of first and second contacts 10, 11. The groups of first and second contacts 10, 11 can be formed as interconnected areas or serpentine portions 21 (not shown). The group of first contacts 10 comprises, for example, three interconnected surface contacts each, while the group of second contacts 11 comprises two interconnected surface contacts.

### LIST OF REFERENCE SIGNS

1 computer device
2 main body
3 inner surface
4 end face
5 detection area
6 interior space/interior of the main body
7 sensor assembly
8 measurement path
9 electrical contacts
10 first contact
11 second contact
12 opposite area
13 vias
14 optical sensor
15 EDA sensor
16 guide
17 printed circuit board
18 processor
19 data interface
20 battery modules
21 serpentine portions

1. A ring-shaped computer device for wearing on the human body, in particular on a finger, which computer device is configured to continuously collect vital parameters and/or bioactive markers of a wearer and make them available to a connected device and comprises:
a ring-shaped main body with an at least partially optically transparent inner surface facing the wearer's skin;
a first sensor for optically detecting vital parameters, in particular a heart rate, via the wearer's skin, wherein the first sensor is separated from the wearer's skin by at least one optically transparent detection area of the inner surface; and
a second sensor for determining the electrical properties of the wearer's skin by means of electrical contacts,
wherein the electrical contacts are applied to the optically transparent inner surface of the main body.

2. The computer device according to claim 1, wherein the electrical contacts are configured to be in contact with the wearer's skin, and wherein the computer device further comprises internal components:
a printed circuit board for receiving the sensors and a data interface;
a processor; and
at least two battery modules for supplying power to the printed circuit board and the processor, which are interconnected to form an overall battery.

3. The computer device according to claim 1, wherein the electrical contacts are formed by an electrically conductive thin film, and/or the main body is optically transparent, and wherein the electrical contacts are applied to the optically transparent main body.

4. The computer device according to claim 1, wherein the main body is formed such that it is transparent to electromagnetic radiation of the wavelengths which are necessary for the measurement principle of the heart rate sensor, in particular a photoplethysmogram (PPG).

5. The computer device according to claim 1, wherein the electrical contacts are arranged distributed over a large area over the entire inner surface of the main body, and wherein the detection area of the first sensor is recessed from the electrical contacts.

6. The computer device according to claim 1, wherein the electrical contacts are formed in a meandering fashion on the transparent inner surface of the main body; and/or wherein the computer device comprises two groups of electrical contacts which are arranged distributed over the inner surface.

7. The computer device according to claim 1, comprising a via in the inner surface of the main body for contacting the internal components, in particular the printed circuit board.

8. The computer device according to claim 1, comprising a guide of the electrical contacts via an end face of the main body into the interior of the main body for contacting the internal components, in particular the printed circuit board, through the electrical contacts.

9. A sensor device for recording vital parameters and/or bioactive markers on the human body, comprising:
a first sensor for optically detecting vital parameters, in particular a heart rate, comprising at least one optically transparent detection area; and
a second sensor for determining electrical properties of the wearer's skin by means of electrical contacts,
wherein the electrical contacts are at least partially applied to the optically transparent detection area of the first sensor.
